SEQUENCE LISTING (19 sequences)

SEQ ID NO: 1 - DNA, 1026 bp, brassica rapa
agacgatggacaactcagctccggactccttacctagatcggaaaccgccgtcacctacgactctccttaccccctctacgcgatgtccttctcctcctccacccaccgaatcgccgtcggaagcttcctcgaagactacaacaaccgcatcgacatcctctccttcgactccgactccatgtccctcaagcccctcccgtccctctccttcgagcacccttaccctcccaccaagctcatgttcagccccccctccctccgccgcagcggcggcggcgacctcctcgcctcctccggcgacttcctccgcctctgggaggtcaacgaagactcctcctccgcggagccagtctccgtcctcaacaacagcaagacgagcgagttctgcgcgccgctgacctccttcgactggaacgacgtcgagccgaagcggttaggcacgtgcagcatcgacaccacgtgcacgatctgggacgtggagaggtccgtggtggagacgcagctcatcgcgcacgayaaagaggtccacgacatcgcgtggggggaggctagggttttcgcctcggtctccgccgacggatcggtgaggatcttcgatctgcgcgacaargagcactccaccatcatctacgagagcccccagcccgatacgccgctcctgaggctcgcctggaacaagcaggacttgaggtgtatggccacgattctgatggattcgaataaggttgtgattctcgatattcgatcgccgacgatgcctgtcgcggagctggagcggcaccaggggagtgtgaacgcgattgcttgggccccgcagagctgtaagcatatctgctcgggtggggatgacgcgcaggctctcatctgggagttgccgacgatggctggaccgaatgggattgatccgatgtcggtttactcggccggttcggagattaaccagctgcagtggtcttcttcgttgcctgattggattggcattgcgtttgctaacaaaatgcagctccttagagtttgaggttcga

SEQ ID NO: 2 - DNA, 931 bp, brassica rapa
agacgatggacaactcagctccggactccttacctagatcggaaaccgccgtcacctacgactctccttaccccctctacgcgatgtccttctccttctcctcccgtccctctccttcgagcacccttaccctcccaccaagctcatgttcagccccccctccctccgccgcagcggcggcggcgacctcctcgcctcctccggcgacttcctccgcctctgggaggtcaacgaagactcctcctccgcggagccagtctccgtcctcaacaacagcaagacgagcgagttctgcgcgccgctgacctccttcgactggaacgacgtcgagccgaagcggttaggcacgtgcagcatcgacaccacgtgcacgatctgggacgtggagaggtccgtggtggagacgcagctcatcgcgcacgacaaggaggtccacgacatcgcgtggggggaggctagggttttcgcctcggtctccgccgacggatcggtgaggatcttcgatctgcgcgacaaggagcactccaccatcatctacgagagcccccagcccgatacgccgctcctgaggctcgcctggaacaagcaggacttgaggtgtatggcgacgattctgatggattcgaataaggttgtgattcttgacattcgatcgccgacgatgcctgtcgcggagctggagcggcaccaggggagtgtgaacgcgattgcttgggcgccgcagagctgtaagcatatctgctcgggtggggatgacgcgcaggctcttatctgggagttgccgacgatggctgggccgaatgggattgatccgatgtcggtttactcggccggttcggagattaaccagctgcagtggtcgtcttcgttgcctgattggattggcattgcgtttgctaacaaaatgcagctccttagagtttgaggttcg

SEQ ID NO: 3 - DNA, 1014 bp, Brassica rapa
atggacaactcagctccggactccttacctagatcggaaaccgccgtcacctacgactctccttaccccctctacgccatgtccttctcctcctccacccaccgaatcgccgtcggaagcttcctcgaggactacaacaaccgcatcgacatcctctccttcgactccgactccatgtccctcaagcccctcccgtccctctccttcgagcacccttaccctcccaccaagctcatgttcagccccccctccctccgccgcagcggcggcggcgacctcctcgcctcctccggcgacttcctccgcctctgggaggtcaacgaagactcctcctccgcggagccagtctccgtcctcaacaacagcaagacgagcgagttctgcgcgccgctgacctccttcgactggaacgacgtcgagccgaagcggttaggcacgtgcagcatcgacaccacgtgcacgatctgggacgtggagaggtccgtggtggagacgcagctcatcgcgcacgacaaggaggtccacgacatcgcgtggggggaggctagggttttcgcctcggtctccgccgacggatcggtgaggatcttcgatctgcgcgacaaagagcactccaccatcatctacgagagcccccagcccgatacgccgctcctgaggctcgcctggaacaagcaggacttgaggtgtatggcgacgattctgatggattcgaataaggttgtgattcttgacattcgatcgccgacgatgcctgtcgcggagctggagcggcaccaggggagtgtgaacgcgattgcttgggcgccgcagagctgtaagcatatctgctcgggtggtgatgacgcgcaggctcttatctgggagttgccgacgatggctggaccgaatgggattgatcctatgtcggtttactcggccggttcggagattaaccagctgcagtggtcgtcttcgttgcctgattggattggcattgcgtttgctaacaaaatgcagctccttagagtttga

SEQ ID NO: 4 - DNA, 1641 bp, brassica rapa
atggattgcgagatctactcaagctcttcttctgaaaaccctagagaccacgtccaatcccttgatctctttcctaacatcactcaaaaccctcataacaacaatacccgaatcgaacctttaccgcttatcgataggatcaacttaaactcaaacctaaacctaaaacctaggccatcgtatgttggcgaaggagatgacgaggtagaagatgaagaggacgttgtagtggacgtgagcttacacatcggccttcctggttccggtaattcaagcaatgggaaagagattgtcacttacgatgccggaaaagacatcgaaaatgaagtttccggcaagtcatattggattccgacggtggatcaaattataataggcttcactcatttttcttgccatgtatgcttcaagacattcaatcgctacaacaatcttcaggtacgaatcattatatattatgcttgttgtgtgtccatgtgcacaaccagatcatatagataaccctatatataattttcttttcttttttttctgagaaattttgcttagaaatatataattttcatatactatttatatctccgtatctaatgttaggattttgttcatacataaaaatatgaattaacttgccaaaagttaaacaaccaatgttaaactaaatgaaaagtccctctggatcttgatatattatcatttttgagtcttattagcttgattaatagtgtgactgaattgttaataacacagaagaccaaagattagattatttattcaacattactaaaaaaaaagattatttattcaacattgaatttttatgtttaataggttttctcatatacaattacattttgttcaatagtaaacaaaatttaatataaattttaagcatatttactcatgagcataaaacactaaattttaaagtcaaaatttatataagagttttaaagacatcaaaacttttttatttaatagttcataccaaaaaagtgtcatgctctatgcagccaaattcaaagaatcgagtctggttataatttcttcagtaaattaccttgatcaatcagtaaaatgattttaaaatttgattcaatgatatcgtgagcatatgttttgaaaatctatcttgaaatcttcaaagcatttcttctatttcattttcgtctaattttgtctttttaacctggttgattccaaatatgaatgcagatgcacatgtggggccacggttcacaatacaggaaaggaccagagtcactgaaagggactcagccaagagccatgcttggcatcccttgttactgctgcgttgaagggtgcaggaaccacatcgatcatcctcggtccaagccactcaaagacttccgaacgctccaaacgcactataagcgcaaacacggccaaaagccttatgcgtgtcgcatttgcggtaagctcttggcagttaagggagattggcgaactcacgagaagaactgtgggaaacgttgggtttgtgtttgcggttccgattttaaacacaaacgctcccttaaagaccatattaaggcttttggacctggtcatgggtcttatccgaccgatttgtttgatgagcactgctcatactcttctgtctctgaaacgctcttttaa

SEQ ID NO: 5 - DNA, 1765 bp, brassica rapa
atggattgcgagatctactcaagctcttcttctgaaaaccctagagaccacgtccaatcccttgatctctttcctaacatcactcaaaaccctaataacaacaatacccgaatcgaacctttaccgcttatcgataggatcaacttaaactcaaacctaaacctaaaacctaggccatcgtatgttggcgaaggagatgacgaggtagaagatgaagaggacgttgttgtggacgtgagcttacacatcggccttcctggttccggtaattcaagcaatgggaaagagattgtcacttacgatgccggaaaagacatcgaaaatgaagtttccggcaaggcatattggattccgacggtggatcaaattataataggcttcactcatttttcttgccatgtatgcttcaagacattcaatcgctacaacaatcttcaggtacgaatcattatacattatgcttgttgtgtgtccatgtgcataaccagatcatatagataaccctatatataattttcttttctttttttttctgagaaattttgcttagaaatatataattttcatatactatttatatctccgtatctaatgttaggactttgttcatacataaaaatatgaattaacttgccaaaagttaaacaaccaatgtttaactaaatgaaaattccctctggatcttgatatattatcttttttgagtcttattagcttgattaatagtgtgactgaattaatgtttaaaaaaacacgtattgttcttagcctgactgtttttttatggtgatgttttgatgcacatatttcatcaatattaatttacgcttgtttcatgtttggaactgaattgttaataacacagaagaccaaagattagattatttattcaacattgctcaaaaaaaaagattatttattcaacattgaatttttatgtttaataggttttctcatatacaattacattttgttcaatagtaaaaaaaattaatataaattttaagcatatttactcatgagcataaaacactaaattttaaagtcaaaatttatataatagttttaaagacatcaaaacttttttatttaatagttcataccaaaaaagtgtcatgctctatgcagccaaattcaaagaatcgagtctggttataatttcttcagtaaattaccttgatcaatcagtaaaaagattttaaaatttgattcaatgataacgtgagcatatgatttgaaaatctatcttgaaatcttcaaagcatttcttctatttcattttcgtctaattttgtctttttacatatgaaaacctggttgattccaaatatgaatgcagatgcacatgtggggccacggttcacaatacaggaaaggaccagagtcactgaaagggactcagccaagagccatgctaggcatcccttgttactgctgcgttgaagggtgcaggaaccacatcgatcatcctcggtccaagccactcaaagacttccgaacgctccaaacgcactataagcgcaaacacggccaaaagccttatgcgtgtcgcatttgcggtaagctcttggcagttaagggagattggcgaactcacgagaagaactgtgggaaacgttgggtttgtgtttgcggttccgattttaaacacaaacgctcccttaaagaccatgttaaggcttttggacctggtcatgggtcttatccgaccgatttgtttgatgagcactgctcatactcttctgtctctgaaacgcacttttaa

SEQ ID NO: 6 - DNA, 1549 bp, Arabidopsis sp.
cacaccaatttgcatcacactatattacccattatttttctacaattatgtggtccacttttcaaatagttcactctcatcataaaccggccggggtccttcaaagctcatgcagttgtgcgttgtaaagatatattgtattccaacaactaattcttgccaaaacacttttggctaatgtcattttcattgaaaacaaagcattataaataggaatgatttgtcatttctggaatggaagaacttgggttttagttaacatttgtgcctaaccaacctctcgtttatgtactaaagcattgtccttgtgattgtgaataaccaatcgctcatttctgtgttagaacagtattgtttattttctgttatttgaatcatttccaatgagaaatgagttacataacacctttatttataaacacaaagatgcacacaaggagaagcaaaatcaataagaagaaaccactccacgtggcccgtcgtcagagttacataattggtgaacgactaaactcagaagaaacaacatgtaataagaaacacagaagcagaagcaaaagccaagcaacatttattttgtcgacaaaaaagccaagaaacattcaatgttgattttgtctaagtaaccaggtgtacatacattacccttaaccatacccaaaatcgtatgtactacgatatgtgtacgtaacagtatctatctacatttatattttataagacattattaaatgggaaacttatagtggctagtggctgctacttggctagcaattaacatcaataatttaataactcaaatgtgaaacatctcattcttctcctttattacaccaaaccatttctcattctttacttaccggtcaggtcaacaattctaccattccccatctttggttaagtttatcttttcattttaaagaaaatatatatgaatgttcatccaataattccaacttcttaaaattgttaatgaacattttaatcaatatataattttaatatcaattttataaataatagaaatgtgaaaaacgcaatttctttgctccttgtagcagtaacacaagtcaaagcaacatttgttttcatttgtttgtcagttagctattttatctacaataatatgttatgctttctgaacaataaataaatatagcattattagatatattcattctgtattttgatttgtccaattagtatatgacacgtctacaagatacatagatctttcattatttcactttctcatccaacgtctggtgaaccaaccattcaaaaatcaaaagtcaacttttaatgcacttgagttttggtcatgcattcatatacatacatatatacatagaagtcattacatgcattatactttacttatgtcaacatgttgcaaaagcactaagatgatatacgtatacatgcatattgcaaaaatcagtggtcccataccattttaagtcatcatgagcgtatgagagtaaattcttctcacatattaataacaacccttcaaagttataagatttttagagagagagctaccacg

SEQ ID NO: 7 - DNA, 18 bp, artificial
  primer
ccgcatgtttcaccaacc

SEQ ID NO: 8 - DNA, 19 bp, artificial
  primer
tggccttacatagtggaag

SEQ ID NO: 9 - DNA, 23 bp, artificial
  primer
atagaaagtaaaggtactctctt

SEQ ID NO: 10 - DNA, 21 bp, artificial
  primer
ggtactctctttttagtgcga

SEQ ID NO: 11 - DNA, 19 bp, artificial
  primer
accagttccttgttcgttc

SEQ ID NO: 12 - DNA, 18 bp, artificial
  primer
gtcccaacctgcgttcta

SEQ ID NO: 13 - DNA, 19 bp, artificial
  primer
cagagcataaatctcctgc

SEQ ID NO: 14 - DNA, 21 bp, artificial
  primer
ccagagcataaatctcttatg

SEQ ID NO: 15 - DNA, 21 bp, artificial
  primer
gtggttgagcgctcagttgca

SEQ ID NO: 16 - DNA, 19 bp, artificial
  primer
tgctcgttcttgacaacac

SEQ ID NO: 17 - DNA, 19 bp, artificial
  primer
gagaattgagagacaaagc

SEQ ID NO: 18 - DNA, 19 bp, artificial
  primer
cacgacgttgtaaaacgac

SEQ ID NO: 19 - DNA, 38 bp, artificial
  primer
cacgacgttgtaaaacgactgctcgttcttgacaacac

(19) United States
(12) Patent Application Publication
(10) Pub. No.: US 2010/0287666 A1
(43) Pub. Date: Nov. 11, 2010

(54) Brassica Rapa Transparent Testa Genes for Controlling Seed Colour in Brassica Species

(76) Inventors: Jiefu Zhang, Winnipeg (CA); Ying Lu, Winnipeg (CA); Zheng Liu, Winnipeg (CA); Muhklesur Rahman, Winnipeg (CA); Peter B.E. Mcvetty, Winnipeg (CA); Genyi Li, Winnipeg (CA)

Correspondence Address: ADE & COMPANY INC., 2157 Henderson Highway, WINNIPEG, MB, R2G1P9, CA

(21) Appl. No.: 12/528,205

(22) PCT Filed: Feb. 21, 2008
(86) PCT No.: PCT/CA2008/000334

§ 371 Date: Jul. 6, 2010

Related U.S. Application Data
(60) Provisional application No. 60/890,885, filed on Feb. 21, 2007.
(60) Provisional application No. 60/948,568, filed on Jul. 9, 2007.

(51) Int. Cl. A01H 1/00; C07H 21/00; C12N 15/82

(52) U.S. Cl. 800/282; 536/24.5; 435/320.1

(57) ABSTRACT

Seed coat color is a very important trait in oilseed type Brassica crops. Identification of the genes controlling the seed coat color is essential to the manipulation of these genes to develop new yellow-seeded germplasm for oilseed breeding The Brassica rapa Transparent Testa Glabra (TTG1) and Transparent Testa (TT1) genes may be used to control seed coat color in Brassica plants.

### PRIOR APPLICATION INFORMATION

The instant application claims the benefit of U.S. Provisional Patent Application 60/948,568, filed Jul. 9, 2007 and U.S. Provisional Patent Application 60/890,885, filed Feb. 21, 2007.

### BACKGROUND OF THE INVENTION

Brassica rapa is a major oilseed and vegetable species throughout the world as well as being one of the parent species of B. napus. Yellow seed coat color is desirable in any oilseed Brassica species because it has been reported that yellow-seeded varieties have a thinner seed coat than black seeded varieties, resulting in comparatively larger endosperm which contributes 5 to 7% more oil in the seed (Liu et al. 1991). The seed meal from yellow seeded varieties also contains higher protein and lower fibre content, which improves the meal quality for poultry and livestock (Shirzadegan and Robelien 1985).

Yellow-seeded varieties in oilseed type Brassica crops, such as ‘Yellow Sarson’ in B. rapa, yellow-seeded B. napus, B. juncea and B. carinata, have inherent advantages over their dark-seeded counterparts in both oil and meal quality (Stringam et al. 1974). Yellow seeds have a significantly thinner seed coat than black seeds, thereby leading to lower hull proportion and higher oil and protein content in Brassica crops. Additionally, some other advantages of yellow seeds involve more transparent oil and lower fiber content in the meal. Consequently yellow seeds result in a better feeding value for livestock (Tang et al. 1997). Hairiness in Brassica species is another important trait that is related to plant defense against insects (Agren and Schemske, 1992).

The inheritance of seed coat color in Brassica species has been analyzed for decades. In B. rapa, Ahmed and Zuberi (1971) reported that a single gene is responsible for the dominant brown seed color of the Indian ‘Toria’ lines over the yellow-seeded ‘Yellow Sarson’ lines. But Stringam (1980) found that brown seed color trait was determined by two independent dominant genes in B. rapa. There are three or four independent recessive genes conditioning yellow seed color trait in B. napus (Liu 1992, Rahman et al. 2001). The hairiness trait is conditioned by a single Mendelian gene in B. rapa (Song et al., 1995) or quantitative loci (QTL) (Nozaki et al., 1997).

Early genetic study by Mohammad et al. (1942) and Jonsson (1975) indicated that three genes are responsible for seed coat color segregation in B. rapa. Later, Stringam (1980) reported that two independent loci controlled seed color and proposed a model for seed coat color genes Brl and Br3. According to Stringam's model, presence of dominant alleles at both loci (Brl and Br3) or presence of dominant alleles only at the first locus (Br]) produce brown seed color, while presence of dominant alleles at a second locus (Br3) and homozygous recessive alleles at the first locus (brl brl) produce yellow-brown seeds. Yellow seeds are produced only when both loci present are in homozygous recessive condition (brl brl br3br3). Schwetka (1982), Zaman (1989) and Ran (2001) confirmed the seed coat color inheritance pattern in B. rapa as proposed by Stringam (1980).

Molecular markers enable marker assisted selection (MAS) permitting selection for a trait at a very early developmental stage. This can significantly reduce the cost of producing breeding lines and can accelerate the breeding program dramatically. There are several molecular markers technologies available for MAS in plant breeding including restriction fragment length polymorphism (KELP), simple sequence repeats (SSR), random amplification of polymorphic DNA (RAPD) (Williams et al. 1990; Karp et al. 1997), amplified fragmentlength polymorphism (AFLP) (Vos et al. 1995), and sequence related amplified polymorphism (SRAP) (Li and Quiros, 2001). The principles of these marker techniques vary and they generate different amounts of information. The SRAP technique is simple and easy to perform, more possibility to amplify ORF or ORF related sequences and selected SRAP PCR products separated on a polyacrylamide gel are easy to sequence (Li & Quiros, 2001). Therefore, the SRAF marker technique was used in this study for the identification of molecular markers linked to seed coat color genes in B. rapa.

Molecular markers, such as restriction fragment length polymorphism (RFLP), random amplified polymorphic DNA (RAPD), amplified fragment length polymorphism (AFLP) and simple sequence repeats (SSR) have been used to map the genes controlling seed coat color in different Brassica species (Teutonico and Osborn 1994, Chen et al. 1997, Somers et al. 2001, Liu et al. 2005). In B. rapa, Teutonico and Osborn (1994) mapped a locus controlling seed coat color on linkage group 5. Bulked segregant analysis (BSA) with AFLPs and SSRs were used to identify markers linked closely to seed coat color trait in B. juncea, and one AFLP marker was converted to an SCAR marker (Negi et al. 2000).

There is limited information about the genes controlling seed coat color in Brassica crops although there are 19 transparent testa (TT) genes, two transparent testa glabra (TTG1 and TTG2), and other genes have been cloned and analyzed functionally in Arabidopsis (Walker et al. 1999, Johnson et al. 2002, Broun 2005; Baudry et al., 2004, 2006). The TTG1 and TTG2 genes control both seed coat color and hairiness in Arabidopsis. Additionally there are several genes, such as glabrous 1, 2, and 3 (GL1, GL2 and GL3) that are demonstrated to involve formation of trichomes (Schiefelbein, 2003). Recently a hairy canola was produced using the Arabidopsis glabrous gene GL3 through genetic transformation (Gruber et al., 2006). To better understand the genes controlling seed coat color and hairiness traits in Brassica crops, a Mendelian locus controlling seed coat color and trichome formation in B. rapa was targeted through map-based gene cloning. SRAP was used to find some molecular markers that were linked to hairiness and seed coat color traits and then chromosome walking was performed with the Arabidopsis genome sequence as a reference.

Several molecular markers linked to seed coat color in Brassica species have been reported. Van Deynze et al. (1995) identified RFLP markers linked to a seed coat color gene in B. napus. Similarly, Somers et al. (2001) developed a RAPD marker for single major gene (pigmentl) controlling seed coat color in B. napus. Zhi-wen et al. (2005) reported that yellow seed color was partially dominant over black seed color and developed 2 RAPD and 8 AFLP markers for the seed coat color gene in B. napus. The RAPD and AFLP markers developed by Zhi-wen et al. (2005) were not suitable for large scale MAS, therefore Zhi-wen et al. (2006) converted these markers into reliable sequenced characterized amplified region (SCAR) and cleaved amplified polymorphic sequence (CAPS) markers for seed coat color breeding in B. napus. Negi et al. (2000) identified an AFLP marker for seed coat color gene in B. juncea and converted the marker into SCAR marker. In another study, SSR markers were developed for mapping and tagging the two independent loci controlling the seed coat colour in B. juncea (Padmaja et al. 2005). Mahmood et al. (2005) identified QTLs associated with the seed coat color in Brassica juncea from an RFLP map using a doubled-haploid population. Chen et al. (1997) identified a RAPD marker linked to a seed coat color gene in a C genome chromosome of a B. campestris-B. alboglabra additional line. Heneen and Jorgensen (2001) identified a RAPD marker on chromosome 4 for brown seed color in B. alboglabra using B. rapa-B. alboglabra monosomic addition lines. To date, no seed coat color gene in B. rapa has been identified. In this study, the inheritance of seed coat color in B. rapa was analyzed using cross progeny from a cross of the self incompatible variety ‘SPAN’ and the self-compatible yellow sarson variety ‘BARI-6’. SRAP, SNP and multiplexed SCAR molecular markers closely linked to a seed coat color gene were developed. These molecular markers will be used for MAS in Brassica breeding and map-based cloning of this seed coat color gene.

### SUMMARY OF THE INVENTION

According to a first aspect of the invention, there is provided a gene-silencing construct comprising at least 20 consecutive nucleotides of the Brassica TTG1 (SEQ ID Nos: 1-3) sequence or TT1 sequence (SEQ ID Nos. 4-5).

According to a second aspect of the invention, there is provided a method of controlling seed color comprising:

transforming a plant with a gene-silencing construct comprising at least 20 consecutive nucleotides of the Brassica TTG1 (SEQ ID Nos: 1-3) sequence or TT1 sequence (SEQ ID Nos:4-5);

growing the plant under conditions whereby the gene silencing construct is expressed, thereby interfering with native TTG1 or TT1 expression such that said plant produces yellow seeds.

### BRIEF DESCRIPTION OF THE DRAWINGS

FIG. 1 Genetic map constructed with 559 DH lines from a cross of glabrous and yellow-seeded and hairy and black-seeded DH parental line in Chinese cabbage (on the left side) and the corresponding region on linkage group R6 of the B. rapa map (on the right side). All markers with SNP are SNP molecular markers; these with SCAR, SCAR markers; and others, SRAP markers.

FIG. 2 Fine map for the region containing the hairiness and seed coat color gene and physical map in the corresponding synteny in Arabidopsis. The physical map was calculated according to the TAIR database. Markers with SNP are SNPs; with SCAR, SCARs; and YQ338 and YB512, SRAPs.

FIG. 3 Multiple amino acid sequence alignment of TTG1 ortholog from black seed of B. rapa and Arabidopsis TTG1. “*” means that the residues or nucleotides in that column are identical in all sequences in the alignment. “:” means that conserved substitutions have been observed. “.” means that semi-conserved substitutions are observed.

FIG. 4. Multiple coding sequence alignment of Ttg1 ortholog from hairless, yellow-seeded (yellow) DH lines, hairy, black-seeded (black) DH lines, and a hairy, black-seeded male sterile line for BAC library construction (BAC-DNA) of B. rapa. “*” means that nucleotides in that column are identical in all sequences in the alignment and others are deletion and SNP positions.

FIG. 5. Seed coat color segregation in the progenies of a cross of yellow-seeded ‘BARI-6’ and ‘brown-seeded ‘SPAN’

FIG. 6. PCR walking from left end and right end of the marker (SA7BG29-245) sequence. Two-step PCR using primer combination APIIMWalk27 and AP21MWalk28 from the left end; and another two-step PCR from the right border with the primer combinations AP1/MWalk24 and AP21MWalk25 were performed. The DNA were taken for first PCR and second PCR from four different genomic libraries constructed by DraI, EcoRV, Pvull and Stul. a. AP1+MWalk27, first round PCR; b. AP2+MWalk28, second round PCR; c. AP1+MWalk24, first round PCR; d. AP2+MWalk25, second round PCR.

FIG. 7. Figure showing SNP detection by GeneScan software (ABI 3100 genetic analyzer) to analyze the SNaPshot Multiplex kit data. The peak information was transformed manually for each loci [e.g. black for ‘C’ and the genotype Br1Brl; red for ‘T’ and genotype brlbri; and black/red for ‘CIT’ and genotype Bribri].

FIG. 8. Multiplexed SCAR marker linked to seed coat color was detected in ABI 3100 genetic analyzer using four different fluorescently labeled primers M13 with unlabeled MR1313 and MR54. The marker linked to the brown seed gene (Br1Br1) produced 388 bp, the yellow or yellow-brown (brlbri) gene generated 400 bp and the heterozygotes (Bribri) produced both 388 bp and 400 bp fragments. a. SCAR marker segregation in the brown-seeded, yellow-seeded and FI genotypes; b. SCAR marker segregation in the F2; c. SCAR marker segregation in the BC1.

FIG. 9. Sequence alignment of span-black and bar1-yellow of TT1.

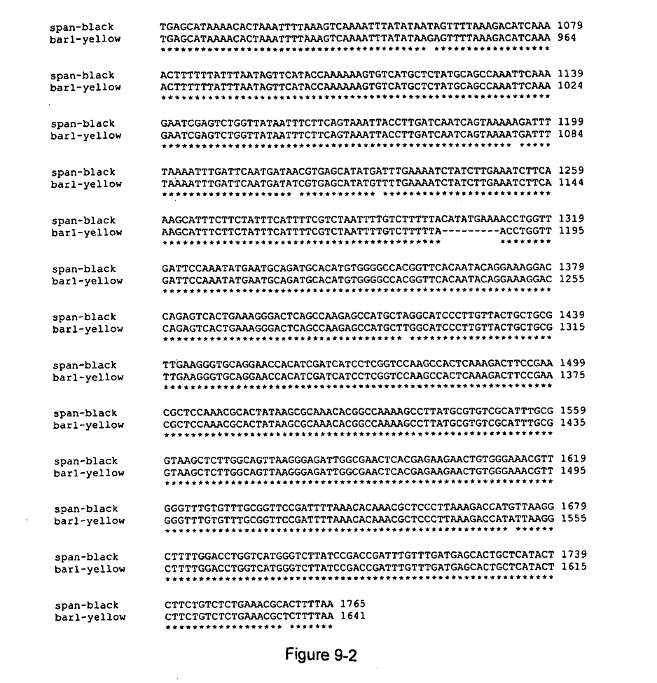

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

The goal of this research was to clone the gene controlling hairiness and seed coat color traits through map-based gene cloning. Since the whole genome sequence in Arabidopsis is available, the close relation of Brassicas to Arabidopsis offers a powerful tool to the Brassica community (Paterson et al. 2001). Since it is easy to sequence SRAP molecular markers and approximately 50% of SRAPs target the gene regions (Li and Quiros, 2001), SRAP molecular markers allow the identification of the corresponding region in Arabidopsis. However, the dissimilarity between the Brassica and Arabidopsis genomes may result in misleading information and the comparative genomics between Brassicas and Arabidopsis should be performed cautiously. For instance, the sequence of the SRAP molecular marker YB512 in this report matched a gene At3g62850 on chromosome 3 in Arabidopsis. Actually the flanking genes of At3g62850 in Arabidopsis are different from the genes surrounding the At3g62850 homolog in B. rapa. Therefore, the chromosome walking in B. rapa with the sequence of the flanking genes of At3g62850 in Arabidopsis is impossible. In this case, a BAC library and more SRAP molecular markers helped solving this difficulty. Closely linked SNP molecular markers were developed that allowed the continuation of the chromosome walking to the final identification of the candidate gene that controls hairiness and seed coat color traits in B. rapa.

Seed coat color is a very important trait in oilseed type Brassica crops. Identification of the genes controlling the seed coat color is essential to the manipulation of these genes to develop new yellow-seeded germ plasm for oilseed breeding. In Arabidopsis, there are more than 20 genes controlling seed coat color. Some of these such as TTG1 and TTG2, also function in the pathway of trichome formation. Some of these genes, such as BANYULS (BAN), TT3, TT4, TT5, TT6, and TT7, encode enzymes in the biosynthesis of flavonoid compounds (Baudry et al. 2004, 2006, Broun 2005). However, others belong to regulatory factors, such as TT1, TT2, TT8, TT16, TTG1 and TTG2 that regulate the expression of enzyme-encoding genes. There is a line of evidence that TT2, TT8 and TTG1 form a tertiary complex that directly activates the expression of other genes, such as BAN (Baudry et al. 2004). Mutation of these three genes leads to yellow seed coat color in Arabidopsis. Combined with the different member of MYB and bHLH transcription factor, TTG1 can also form a complex that regulates trichome initiation, mucilage formation and root hair spacing. Fortunately a TTG1 homolog was identified in this report that functions both in trichome formation and seed coat color. The effects of this gene on mucilage biosynthesis and root hair spacing have not yet been studied. Therefore, analysis of the DH line population to determine the mutation effect of TTG1 homolog in B. rapa on mucilage and root hair spacing is planned. If these two traits change as seen in Arabidopsis, it would provide even more convincing evidence that the candidate gene in B. rapa found in this report functions exactly as it does in Arabidopsis.

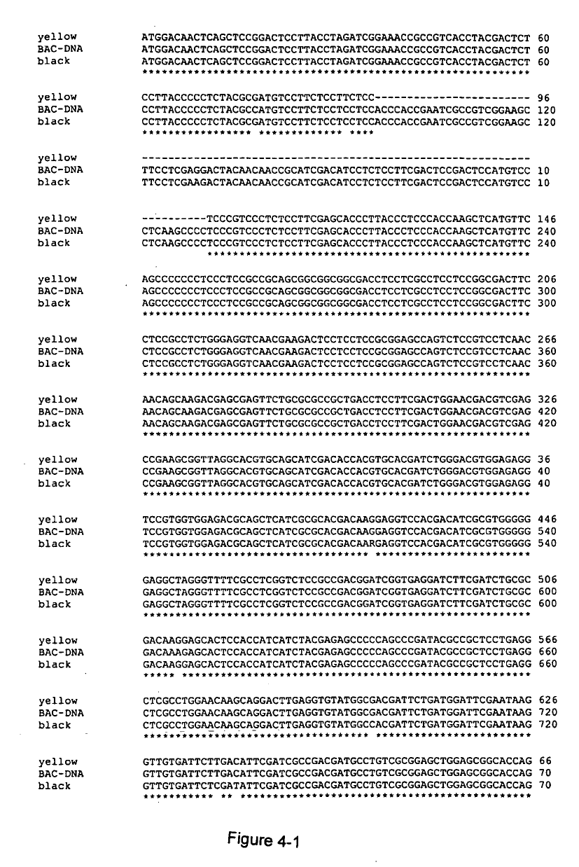

The hairless, yellow-seeded parental line for producing the DH mapping population is a natural recessive mutation. The comparison of sequences from hairless, yellow-seeded and hairy, black-seeded materials led to identification of a deletion in the hairless, yellow-seeded materials, clearly indicating that the mutation contributes to a nonfunctional truncated protein. This is a common case if a deletion happens in an open reading frame. The TTG1 gene codes for a WD-40 repeats protein with a a helix at the N terminal and over a dozen β sheets spreading the rest part of the protein (Walker et al. 1999). Compared with Arabidopsis TTG1, the Brassica TTG1 (SEQ ID Nos: 1-3, wherein SEQ ID No. 1 encodes the black seed, SEQ ID No. 2 encodes the yellow seed and SEQ ID No. 3 is from the BAC preparation) ortholog shared nearly identical functional domains (FIG. 4). Although there is a four-amino acid deletion located in the a helix, most of the other changes belong to conserved or semi-conserved substitutions, indicating that the Brassica ortholog codes for the same protein as that of the TTG1 per se and these two proteins function in similar fashion.

The B. rapa yellow sarson parent line variety ‘BARI-6’ (SEQ ID No. 4, yellow seeds) was taxonomically different from the Canadian B. rapa parent line variety ‘SPAN’ (SEQ ID No. 5, black seeds). Yellow sarson belongs to ssp. trilocularis and is self-compatible, while ‘SPAN’ belongs to ssp. oleifera and is self-incompatible. Using a self-compatible parent in the cross made it easier to self plants in the greenhouse. A pollen effect was observed when yellow sarson was used as the female parent, resulting in dark yellow F1 seeds instead of bright yellow FI seeds. This is known as a Xenia effect in yellow sarson and could be used as an indicator for successful crosses. This phenomenon was also observed by Rahman et al. (2001) who used an open pollinated yellow-seeded B. napus line that was derived from yellow sarson, suggesting that yellow sarson contains the gene(s) for Xenia effect.

Digenic inheritance with dominant epistasis was observed for seed coat color segregation in B. rapa. The dominant epistatic gene was responsible for brown color and the hypostatic gene was responsible for yellow-brown seed color, and yellow seed color was observed when both the genes were in homozygous recessive condition. These results confirm the seed coat color segregation results reported by Stringam (1980) and by Rahman (2001).

A dominant SRAP marker is less convenient than a co-dominant marker for large scale MAS in plant breeding. Consequently, the dominant SRAP marker developed in this study was converted to co-dominant SNP or SCAR markers, following the lead of several researchers who converted their dominant markers into co-dominant markers, such as SCAR marker from RAPD markers (Naqvi and Chattoo 1996; Lahogue et al. 1998; Barret at al. 1998) and AFLP markers (Negi et al. 2000; Adam-Blondon at al. 1998; Bradeen and Simon 1998), and SCAR and CAPS markers from RAPD and AFLP markers (Zhi-wen et al. 2006). There was no difference between brown-seeded and yellow-seeded lines in the 214 by sequence of the SRAP marker. A single nucleotide polymorphic position is required for the development of co-dominant SNP markers. Co-dominant SCAR markers are developed from the insertion or deletion fragments position in any of the two sequences. Even development of CAPS markers required the DNA fragments size range of 500 to 1500 by (Barrett et al. 1998). Therefore, a 214 by SRAP sequence limits the development of any co-dominant SNP, SCAR or CAPS markers. However, the extended flanking sequence from the SRAP marker allowed the development of SCAR or SNP co-dominant markers. Chromosome walking approach was used to obtain the flanking sequence adjacent to the SRAP marker. It had been proven that chromosome walking is one of the best methods for having the flanking sequence adjacent to a sequence of interest (Devic et al. 1997, Negi at al. 2000). Negi at al. (2000) successfully converted the AFLP markers to the SCAR markers using chromosome walking method and isolated the large-sized fragments adjacent to the AFLP markers which did not require any optimization for different walking. We obtained more than 1.8 kb flanking sequences from the SRAP markers that showed 24 SNPs and a 12 by deletions or a 12 by insertions site which allowed developing SNP markers and SCAR markers, respectively.

The SNaPshot method used in this study is simple, requires very little optimization and is high throughput using an ABI 3100 genetic analyzer (Nirupma at al. 2004). SNP markers are co-dominant, and have been found more abundant in genomic sequences that can potentially be used for MAS. The SNP markers developed in this study used to screen the F2 and BC1 generations showed the same pattern as the SRAP marker, indicating that the SRAP marker was successfully converted into SNP markers that were closely linked to the Br9 seed coat color gene. The major shortcoming of the SNP marker approach is cost.

A cost effective alternative to SNP markers are SCAR markers, most especially multiplexed SCAR markers. In this study, a 12-bp deletion in the brown seeded lines allowed the development of multiplexed co-dominant SCAR markers. Here we used four fluorescently labeled M13 primers with single unlabeled primer that allowed pooling four PCR products for the detection in an ABI 3100 genetic analyzer (four fluorescently labeled M13 primers were universally used to combine with any co-dominant multiplexing SCAR markers in our laboratory). However, in principle, any primers covering this 12 by deletion region would produce two bands with a 12 by sequence difference. Using the M13 primer labeled with four fluorescent dye colors and a series of primers that produced fragments 12-bp different in length permitted the pooling of several hundred amplified DNA samples for signal detection using the ABI Genetic Analyzers. Multiplexed SCAR markers can reduce the running cost of the ABI DNA Genetic Analyzer dramatically and significantly increase the efficiency of MAS in a breeding program compared to the high cost of SNP detection. For example, we designed 20 unlabeled primers to target a two-base deletion position in the Bn-FAE9-2 gene of the C genome of B. napus and combine with a genomespecific primer that was labeled with four fluorescent colors to form 80 primer pairs in total, and each primer pair was used to amplify different DNA samples. After PCR, 80 samples were pooled and 1280 (16×80) samples was analyzed with an ABI 3100 Genetic Analyzer in 40 minutes (unpublished data). The running cost was reduced by 80 times compared with that of SNP detection with the ABI SNaPShot detection kit. Actually more unlabeled primers could be designed to increase the pooled samples to reduce the cost further. Therefore, multiplexing any co-dominant SCAR markers targeting deletions or insertions (INDELs) has great potential for MAS in plant breeding if a sample pooling strategy as described in this report is implemented.

As discussed above, the yellow-seeded varieties of oilseed crops have inherent advantages over their dark-seeded counterparts in both oil and meal quality. Accordingly, in one aspect of the invention, there is provided a gene-silencing construct comprising at least 20, at least 25, at least 50, at least 75, at least 100 or at least 200 consecutive nucleotides of the Brassica TTG1 (SEQ ID Nos: 1-3) sequence or TT1 sequence (SEQ ID Nos 4-5). As will be appreciated by one of skill in the art, the nucleotide sequence may be derived from the sense or anti-sense of TTG1 or TT1. In one embodiment, the construct is an RNAi construct, although as will be appreciated by one skilled in the art, other suitable silencing constructs known in the art may also be used. It is further of note that such silencing constructs and their use in Brassica are well known, see for example Zhi et al., 2006 Journal of Plant Physiology and Molecular Biology 32: 665-671; Wesley et al., 2001, The Plant Journal 27: 581-590; Jadhav et al., 2005, Metab Eng 7: 215-220. Specifically, RNAi constructs have been made with sequences of Brassica TT1 and TTg1 homologs to transform canola. More than 50 transgenic plants were produced for each construct. Initial data has shown that at least some seeds of some transgenic plants with Brassica TT1 homolog construct showed seed coat color change and transgenic plants with the Brassica TT1 homolog construct started flowering.

As discussed herein, any suitable promoter may be used in the preparation of silencing constructs. Such promoters will be readily apparent to one of skill in the art. In one embodiment of the invention, four seed coat—specific promoters from Arabidopsis were tested with a functional copy of Brassica TTG1 homolog and it was found that the upstream sequence of TT8 (SEQ ID No. 6) driving Brassica TTG1 homolog in Arabidopsis changed yellow seeded coat color of a ttg1 mutant into black seeded one.

Furthermore, it is of note that given the high degree of identity across Brassica species, silencing constructs will work across Brassica species.

The natural seed coat color for all canola cultivars and most other Brassica oilseed crops is black. Since yellow-seeded Brassica oilseed crops increase seed oil and protein content (and are better than black-seeded ones), through gene silencing, the TTG1 homolog in Brassica rapa here will be used to develop yellow-seeded lines in any Brassica species that can be used in breeding. These new lines will be totally yellow-seeded.

According to a second aspect of the invention, there is provided a method of controlling seed color comprising:

transforming a plant with a gene-silencing construct comprising at least 20, at least 25, at least 50, at least 75, at least 100 or at least 200 consecutive nucleotides of the Brassica TTG1 (SEQ ID Nos: 1-3) sequence, shown in FIG. 4, or TT1 (SEQ ID Nos. 4-5);

growing the plant under conditions whereby the gene silencing construct is expressed, thereby interfering with native TTG1 or TT1 expression such that said plant produces yellow seeds.

Preferably, the plant is a Brassica species.

In another embodiment of the invention, there is provided a method of using the sequences described above for developing yellow seeded Brassica plant through marker development and marker-assisted selection.

In another embodiment of the invention, there is provided a method of using an expression construct comprising the seed-specific tt8 promoter (SEQ ID No. 6) described above operably linked to the sequence of Brassica TTG1 (SEQ ID No. 2) or TT1 (SEQ ID No. 4) homologs to produce a yellow seeded Brassica plant. As will be appreciated by one of skill in the art, a suitable expression construct comprising the tt8 promoter operably linked to the sequence as set forth in SEQ ID No. 2 or SEQ ID No. 4 may be prepared and introduced into a suitable Brassica plant. The plant may then be grown under conditions suitable for expression from the tt8 promoter, thereby producing yellow seeds.

### Results

Mapping the Gene Controlling Hairiness and Seed Coat Color Gene with SRAP and SCAR Markers

F1 plants of the reciprocal crosses between a hairy, black-seeded parent, ‘Y195-93’, and a glabrous, yellow-seeded parent, ‘Y177-12’, were hairy and the seeds on the F1 plants were black, indicating that hairy and black-seeded traits were dominant over glabrous and yellow-seeded traits. Among 559 DH lines that were used for gene tagging, 254 DH lines were hairy and black-seeded while 305 DH lines were glabrous and yellow-seeded. Therefore, the hairiness and seed coat color traits co-segregated completely in this mapping population and the segregation ratio of the glabrous, yellow-seeded lines versus hairy, black-seeded lines was 1:1 (X², p=0.086), suggesting that one Mendelian locus controlled both hairiness and seed coat color in this population.

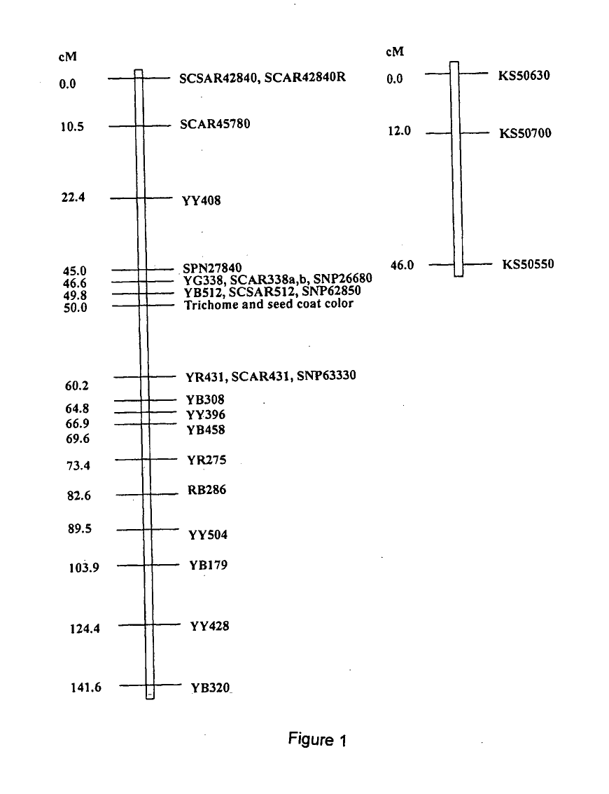

Using a BSA strategy, 1100 SRAP primer combinations were used to amplify four DNA bulks from 16 (4×4) glabrous and yellow-seeded DH lines and 4 others from 16 (4×4) hairy and black-seeded lines. After observing the polymorphism, 48 out of the SRAP 1100 primer combinations were selected to amplify 16 glabrous and yellow-seeded and 16 hairy and black-seeded DH lines. Then 13 out of the 48 primer pairs were found to produce polymorphic loci that were linked to hairiness and seed coat color. These thirteen SRAPs were used to analyze the whole mapping population and a genetic map was constructed for the region containing the hairiness and seed coat color gene (FIG. 1).

The corresponding bands to seven SRAP markers, YG338, YB512, YR431, YYb197, YY396, YB458 and YB308, were cut from polyacrylamide gel and DNA was recovered and sequenced. After BLAST analysis with TAIR Arabidopsis database (http://www.arabidopsis.org), four of them were found to have a match to the annotated genes in Arabidopsis. The sequences of YG338, YB512, YR431 and YYb197 corresponded to the Arabidopsis genes AT5G26680, AT3G62850, AT5G63330 and AT2G19110, respectively.

New primers JF39 (SEQ ID No. 7) and JF40 (SEQ ID No. 8) were designed using the sequence of SRAP marker YR431, and were used to amplify DNA from 4 glabrous and yellow-seeded and 4 hairy and black-seeded DH lines for sequencing. JF39 (SEQ ID No. 7) and JF40 (SEQ ID No. 8) produced different sized fragments between glabrous, yellow-seeded and hairy, black-seeded DH lines. After sequencing, a 93-bp deletion was found between the fragments from glabrous, yellow-seeded DH lines (340 bp) and hairy, black-seeded ones (247 bp). Therefore, the SRAP molecular marker YR431 was converted to a co-dominant SCAR marker SCAR431 that was integrated into the map (FIG. 1).

Primers JF5G3 (SEQ ID No. 9) and JF5G4 (SEQ ID No. 10) designed using the sequence of SRAP marker YG338, were used to amplify DNA from glabrous, yellow-seeded and hairy, black-seeded lines, but no DNA fragment difference between these DH lines was found. They were used to select a BAC clone A73M7 from a B. rapa BAC library. BAC end sequencing was performed and BLASTn analysis showed that one end sequence matched an Arabidopsis AT5G26680 gene, the same gene matched by the sequence of SRAP marker YG338, while the other end did not match any gene in Arabidopsis. Since the end sequence matched the same gene as the sequence of the marker YG338, the SRAP marker YG338 was located at the end of BAC clone A73M7. With the new end sequence, another pair of primers JF5G5 (SEQ ID No. 11) and JF87a (SEQ ID No. 12) were designed to amplify DNA from glabrous, yellow-seeded, and hairy, black-seeded DH lines, and SNPs were discovered. Two new primer pairs, JF106 (SEQ ID No. 13) and JF106b (SEQ ID No. 14) located at the SNP positions were designed. Interestingly, JF106 (SEQ ID No. 13) and JF87a (SEQ ID No. 12) produced a band in glabrous and yellow-seed DH lines, whereas JF106b (SEQ ID No. 14) and JF87a (SEQ ID No. 12) amplified a band in hairy and black-seeded DH lines. These two dominant SCAR markers produced a co-dominant SCAR marker when they were used separately. These markers named SCAR338a and SCAR338b were integrated into the map (FIG. 1).

All SRAP molecular marker sequences mentioned previously were analyzed with BLAST server on the website of Brassica genome gateway and some of these SRAP markers matched sequences of B. rapa BAC clones on the genetic map and physical map. Primers were designed according to the B. rapa BAC sequences to identify new SNPs. Among these new SNPs, four SNPs were successfully converted to SCAR markers. These were SCAR27840, derived from B. rapa BAC KBrS016J18, SCAR42840 and SCAR42840R, derived from BAC KBrB061E18, and SCAR45780, derived from BAC KBrH003E13. SCAR27840 and SCAR42840R were dominant SCAR markers with bands in glabrous, yellow-seeded DH lines. SCAR42840 was dominant, showing a band in hairy, black-seeded DH lines, while SCAR45780 was a co-dominant SCAR marker. After testing with the segregating DH line population, these four SCAR markers were integrated into a linkage group (FIG. 1). Each of these three B. rapa BAC clones had a corresponding molecular marker, which were KS50630, KS50700 and KS50550, located on linkage group 6 (R6) of the B. rapa genetic map. The map distance of these four SCAR markers on the current map nearly covered the same genetic distance as that of the markers for the BAC clones on the map (FIG. 1).

### Identification of the Candidate Gene for Hairiness and Seed Coat Color Traits

YB512 was the SRAP molecular marker on the map that was most closely linked to the hairiness and seed coat color gene, and the sequence of this marker matched a gene AT3G62850 on Arabidopsis chromosome 3. Using the gene sequence from the flanking region of At3g62850 in Arabidopsis, several primer pairs were designed to amplify DNA from 4 glabrous, yellow-seeded and 4 hairy, black-seeded DH lines. After sequencing, SNPs were identified, but unfortunately these SNPs did not co-segregate with the hairiness and seed coat color traits in the DH population. Thus the gene order around the SRAP marker YB512 in Chinese cabbage was not conserved with regard to the corresponding Arabidopsis gene order in this region. Consequently the chromosome walking with At3g62850 could not be performed further.

The primers designed with the sequence of SRAP marker YB512 could amplify DNA from glabrous and yellow-seeded DH lines, but not from hairy and black-seeded lines. Since the material used for the B. rapa BAC library construction was hairy and black-seeded, all the primers designed with the sequence of the marker YB512 were not able to produce a band in the hairy, black-seeded B. rapa lines and were therefore, not adequate for screening the B. rapa BAC library. To continue the chromosome walking with this closely linked marker, genome walking was used to extend the sequence of the SRAP marker YB512 to its flanking regions and 1 kb of extra sequence outside the marker in Chinese cabbage was obtained. With the genome walking sequence, new primers were designed to amplify DNA from glabrous and yellow-seeded and hairy and black-seeded DH lines, and new SNPs were identified. These SNPs were found to co-segregate with hairiness and seed coat color traits. Meanwhile, these new primers allowed the selection of a BAC clone A6L12 from the B. rapa BAC library. The BAC ends of A6L12 clone were sequenced and BLAST analysis with TAIR database showed that the end sequences of the BAC A6L12 had a match to AT5G24690 and AT5G24650 on Arabidopsis chromosome 5, respectively. Since the sequence from another closely linked SRAP marker YG338 matched AT5G26680, and the SNP27840 developed with the B. rapa BAC clone sequence matched AT5g27840, both of which were located at the syntenic region on chromosome 5. Consequently a region on Arabidopsis chromosome 5 was confirmed to be the real synteny, not the region on chromosome 3 that was suggested with the sequence from the closest linked marker YB512.

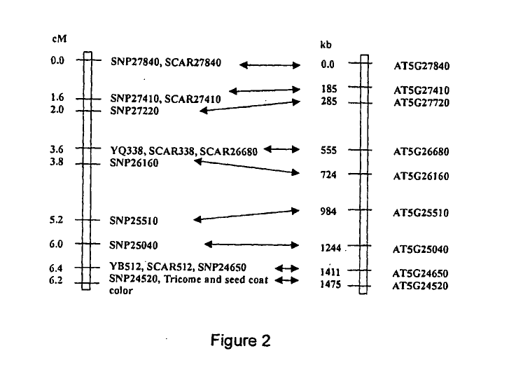

The following chromosome walking focused on the syntenic region on Arabidopsis chromosome 5. SNPs were discovered in B. rapa homologs of AT5G27410, AT5G27220, AT5G26680, AT5G26160, AT5G25510, AT5G25040, AT5G24650, and AT5G24520 on Arabidopsis chromosome 5, and some of these SNPs were also converted to SCAR markers. After testing these SNPs and SCARs with the mapping DH line population, the data showed that, with the exception of the gene matched by the SRAP marker YB512, all others corresponded to these SNPs and SCARs in B. rapa were in the same order as that in Arabidopsis. The SNP inside the Brassica homolog of At5g24520 (TTG1) in Arabidopsis showed no recombination with the hairiness and seed coat color gene (FIG. 2). These results suggested that the gene in B. rapa is a TTG1 homolog that functions exactly the same in both B. rapa and Arabidopsis with respect to hairiness and seed coat color traits.

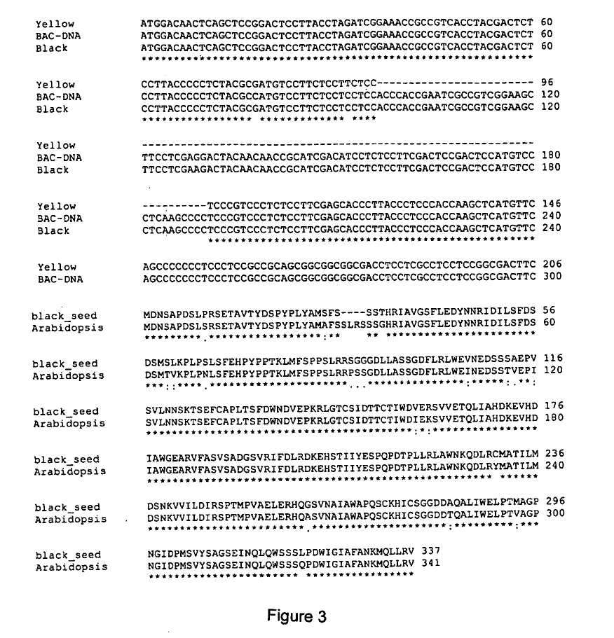

After finding the candidate gene, a BAC clone anchoring the TTG1 orthologous gene was selected from a B. rapa BAC library that was constructed with a hairy, black-seeded male sterile line. With primer walking, the whole sequence of the TTG1 orthologous gene was produced. New primers were designed to amplify the coding sequences of the TTG1 ortholog from two alleles in hairless, yellow-seeded and hairy, black-seeded DH lines were analyzed. After Clustalw analysis, a 94-base deletion was detected in the hairless, yellow seeded DH lines (FIG. 3), but there were only a few base changes between the sequences from the hairy and black-seeded DH lines and the material used for the BAC library construction. Compared with Arabidopsis TTG1, the starting codon and stop codon for three sequences of the TTG1 ortholog in B. rapa. The deduced amino acid sequence from the hairless and yellow-seeded DH lines showed several stop codons and a truncated protein was produced (data not shown), suggesting that this nonfunctional protein resulted in a hairless, yellow-seeded natural mutant. These two sequences from the hairy, black-seeded DH line and the line for the BAC library shared the same protein sequence containing 337 amino acids. With Clustalw analysis, it was found that the protein of the Brassica TTG1 ortholog was 93% identity to Arabidopsis TTG1 (FIG. 4). Between these two proteins, there was a deletion of four amino acids corresponding to the 31-34 amino acid positions in Arabidopsis TTG1 gene and most of other changes were conserved or semi-conserved substitutions.

### Seed Coat Color Inheritance in B. Rapa

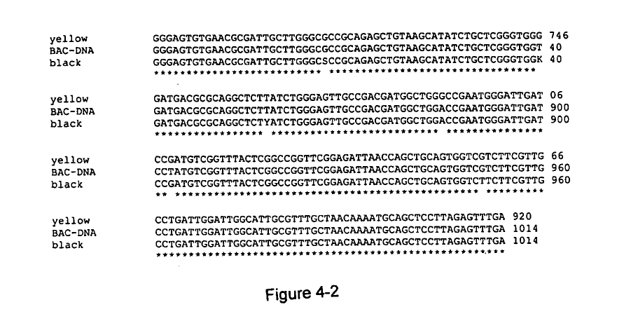

Inheritance of seed coat color was analyzed using 224 F2 individuals from a cross of ‘SPAN×BARI-6’. It was found that seed coat color was mainly controlled by the maternal genotype; therefore the F1 produced brown color when a brown-seeded variety was used as the maternal parent. A pollen effect was observed when yellow sarson was used as the female parent so the FI seed coat color was dark yellow instead of bright yellow. Seed coat colors in F2 segregated into brown, yellow-brown and bright yellow color (FIG. 5), indicating incomplete dominance of the brown color as described by Shirzadegan (1986). Of 224 F2 plants, 164 were co-segregated with the brown color, 48 were co-segregated with the yellow-brown color and 12 were co-segregated with the bright color. The Chi-square test showed that the seed coat color segregated in a ratio of 12:3:1 (x2=1.238, P=0.5-0.7), confirming digenic inheritance of the trait. However, when 164 brown seeded lines were placed in one group and 60 yellow-brown and bright yellow seeded lines were placed in another group, seed coat color segregated in a monogenic inheritance pattern (x2=0.381, P=0.5-0.7).

Self pollinated seeds of 197 BC1 plants from the [(SPAN×BARI-6)×BARI-6] cross were also used for seed coat color segregation analysis. The seed coat colors in BC1 also segregated into brown, yellow-brown and bright yellow classes. Of the 197 BC1 plants, 95 had brown seed color, 55 had yellow-brown seed color and 47 had bright yellow seed color plants. Chi-tests showed that the progenies fit a digenic (2:1:1, x2=0.898, P=0.5-0.7) segregation ratio for seed coat color. However, when 95 brown seeded lines were placed in one group and 102 brown yellow and bright yellow were placed in another group, seed coat color appeared to segregate in a monogenic manner (1:1, x2=0.248, P=0.5-0.7).

### SRAP Molecular Markers for Seed Coat Color

Forty eight different SRAP primer pairs were used for the development of molecular markers for the seed coat color trait in B. raga. Initially, sixteen brown-seeded lines and sixteen bright yellow-seeded lines from BC1 population were used for the identification of molecular markers using all 48 primer combinations. The markers SA7BG29-245, ME2FC1 266, FCI BG69-530, PM88PM78-435, SA12BG18-244 and SA12BG38-306 were found to be linked to the seed coat color with few recombinants. After testing these markers using the F2 and BC1 generations, the marker SA7BG29-245 was found to be closely linked to seed coat color. There were two recombinant alleles among the total 448 ones of these 224 F2 plants that was equal to a genetic distance of 0.47 cM between the molecular marker SA7BG29-245 and the seed coat color trait, and two recombinant alleles among the total 197 ones that came from the FI plants for producing these 197 BC1 plants, resulting in a genetic distance of 1.02 cM in the BC1 population.

### Chromosome Walking and SNP Development

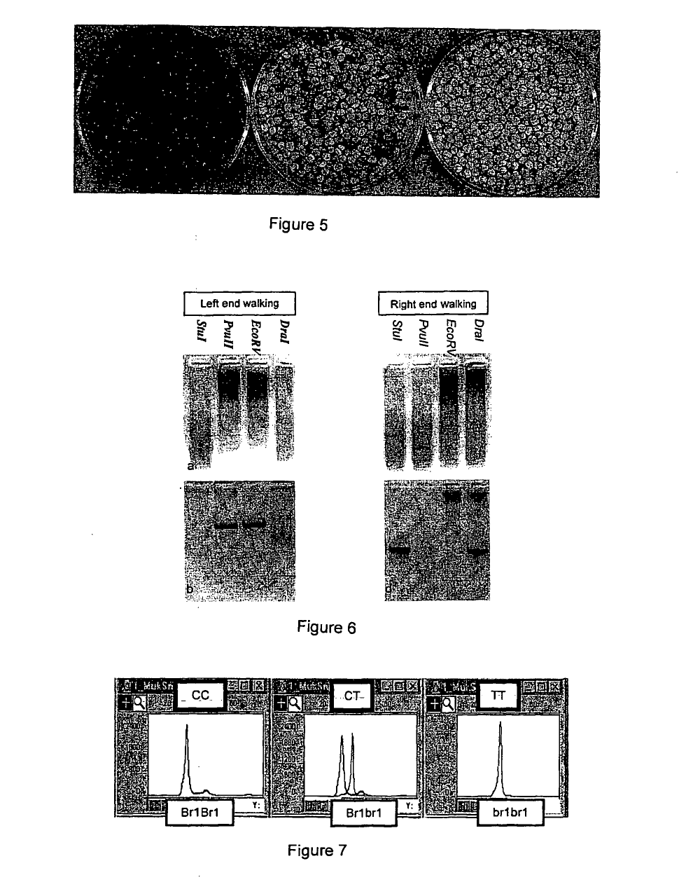

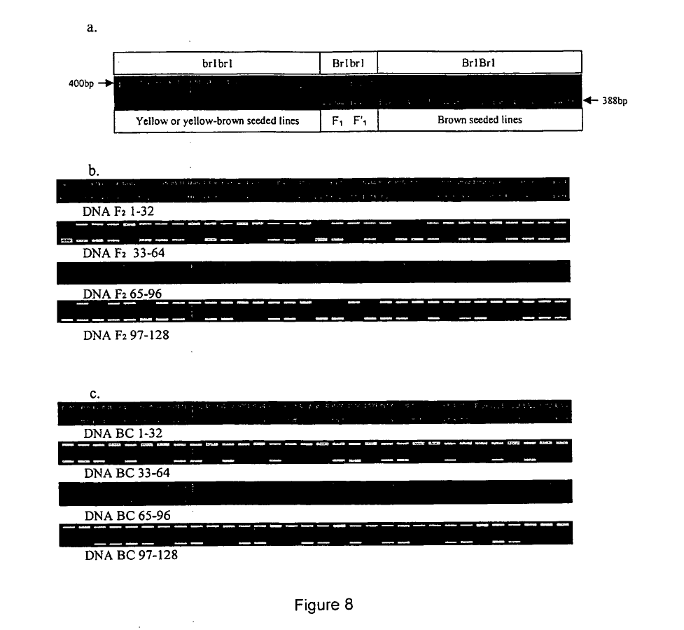

The SRAP molecular marker SA7BG29-245 was sequenced and its flanking 30 sequences were obtained by chromosome walking. Two-step PCR reactions were performed. The first PCR amplification using the left side marker specific primer MWalk27 and adaptor specific primer API produced a smear in all lanes (FIG. 6a). The second PCR amplification using the adaptor specific primer AP2 and marker specific primer MWalk28 produced a single strong band with EcoRV and Pvull (FIG. 6b). Similarly, the first PCR amplification using the adaptor specific primer API and marker specific primer MWalk24 from the right end generated a smear in all lanes (FIG. 6c); and the second PCR amplification using the adaptor specific primer AP2 and marker specific primer MWalk25 generated two strong bands with Drat and Stul (FIG. 6d). A total of 529 bp was extended from left end and 427 bp from right end and in total an 1170 bp fragment was obtained from brown seeded variety ‘SPAN’ (GenBank Accession Number EF488953, EF488954). Unfortunately, the sequence did not match any gene in Arabidopsis after BLAST analysis against the Arabidopsis database. After sequencing the corresponding region in the yellow-seeded parent, 24 SNPs were found between the brown-seeded and yellow-seeded parent lines (SI in supplementary material). The SNPs were detected with an ABI SNaPshot Multiplex kit. For example, one SNP position (at 1041 by position of ‘SPAN’) for homozygous brown seed color was ‘C’ and generated a black peak, heterozygous plants, ‘CIT’, generated both a black peak and a red peak, and homozygous yellow-brown or bright yellow seed coat color, T, generated a red peak (FIG. 7). Since the marker was closely linked to the major seed coat color gene Brtlbrl, the black peak identified homozygous brown seed color Brl Brl genotypes; the dual black and red peaks identified heterozygous brown seed color Br1 br1 genotypes; while the red peak identified homozygous bright yellow or yellow-brown seed color brlbrl genotypes. The SNP markers were tested using both the F2 and BC1 generations, and were found to be at the same genetic distance (0.47cM) from the seed coat color gene as the SRAP molecular marker SA7BG29-245.

### Development of Multiplexed SCAR Markers

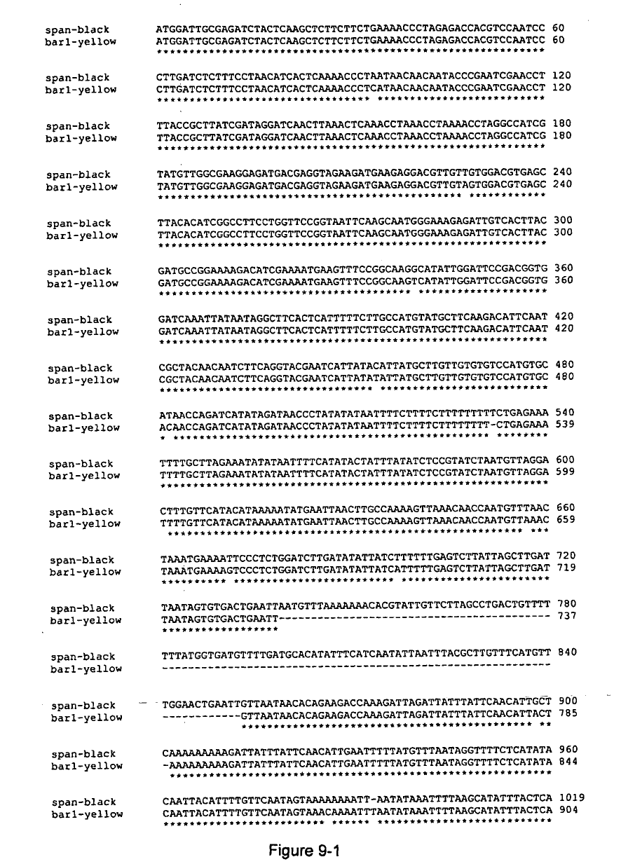

On the basis of 1170 by for the SRAP marker and its flanking sequences, no deletion or insertion polymorphic region was found between brown and yellow seeded lines. Therefore, chromosome walking was performed again to obtain additional extended flanking sequence from the left side. With the new chromosome walking sequence, a 12-bp deletion in the brown seeded lines or a 12-bp insertion in the yellow-brown or bright yellow seeded lines was identified, which were used for the development of multiplexed SCAR markers. Primers MR1313 and MR54 were designed to target the 12-bp deletion. Together with the 19-bp M13 sequence, a 388-bp fragment for brown seeded lines and a 400-bp fragment for yellow-brown or bright yellow seeded lines were produced, respectively (FIG. 8). Since the SCAR marker was not far from the SRAP marker and SNPs mentioned previously, the genotyping of the SCAR marker in 224 F2 plants and 197 BC1 plants were exactly the same as that of the SRAP and SNP markers.

### Materials and Methods

A cross of a hairy, black-seeded B. rapa Chinese cabbage DH line, ‘Y195-93’, and a glabrous, yellow-seeded B. rapa Chinese cabbage DH line, ‘Y177-12’, was used to produce 559 DH lines through microspore culture. These DH lines of B. rapa were used for gene tagging.

Genomic DNA was extracted using a modified 2xCTAB method as described by Li and Quiros (2001). SRAP PCR reactions were set up using the same components and amplification program as reported by Li and Quiros (2001). The SRAP PCR products were separated with ABI 3100 Genetic Analyzer (ABI, California) using a five-color fluorescent dye set, including ‘FAM’ (blue), ‘VIC’ (green), ‘NET’ (yellow) and ‘PET’ (red), and ‘LIZ’ (orange as the standard). Samples from four different color labeled primers were pooled together after running PCR reactions and 2.5 μl of the pooled samples was added to a 5.5 μl mixture of formamide and 500-LIZ size standard (ABI), and then denatured at 95° C. for three minutes. The plates containing the samples were then loaded into the auto sampler of the ABI 3100 Genetic analyzer.

The gene controlling hairiness and seed coat color was first tagged with bulk segregant analysis (BSA) (Michelmore et al., 1991). Equal quantities of DNA from glabrous, yellow-seeded and hairy, black-seeded DH lines were pooled to create DNA bulks. The DNA bulks were subjected to SRAP analysis to identify putative markers linked to the hairiness and seed coat color gene. Then the candidate SRAP markers were used to analyze the whole population.

Some SRAP markers that were linked to hairiness and seed coat color traits were sequenced via the following protocol. Denatured polyacrylamide gels were used to separate SRAP PCR products. After electrophoresis, the DNA in gels was colored with a silver staining kit (Promega, Madison, Wis.). The gel pieces containing the selected bands were cut and put into a 1.5-ml eppendorf tube, and 550 μl DNA elution buffer (500 mM NH₄oAc, 10 mM Mg(oAc)₂, 1 mM EDTA, 0.1% SDS) was added (Sambrook and Russell, 2001). After incubation at 37° C. with shaking at 200 rpm for 24 hours, eluted DNA was precipitated with ethanol and used as template for checking the fragment size with the ABI 3100 Genetic Analyzer. The PCR products with the same size as the SRAP markers were sequenced directly with ABI 3100 Genetic analyzer.

When SRAP markers were sequenced, new primers based on the sequence were designed to amplify 4 yellow seeded and 4 black seeded lines. If there were more than two bases that were different between glabrous, yellow-seeded and hairy, black-seeded DH lines, specific primers were designed on the basis of these sequence differences. The SRAP markers were converted to sequence characterized amplified region (SCAR) markers.

Each polymorphic locus was scored as a dominant marker. Linkage analysis was performed on segregation data of all molecular markers and hairiness and seed coat color traits in the 559 DH lines using Mapmaker version 2.0 for Macintosh (Lander et al. 1987).

A BAC library was constructed following the protocol (Woo et al. 1994). A B. rapa male sterile line was used and a BAC cloning vector, pCCB1 BAC, was purchased from Epicentre (Madison, Wis.). After transformation into E. coil ElectroMAX DH10B (Invitrogen, Toronto, Ontario), colonies were picked up and put into 384-well plates with a QBot robotic system (Genetix, New Milton, U.K.). PCR-based screening of the BAC library was performed with plate pools, column and row pools using a robotic liquid handling system (Tecan, Toronto).

BAC end sequencing was performed with vector primers and primer walking was done directly with BAC clone DNA, following the BAC sequencing protocol in the ABI sequencing kit. After the whole gene sequence of the TTG1 ortholog in B. rapa was obtained through primer walking with the selected BAC clone, new primers were designed to amplify the corresponding copies from both hairless, yellow-seeded and hairy, black-seeded DH lines. Sequence comparison was performed with Claustalw software. The sequence of TTG1 was taken from TAIR database.

Some primers used are listed in Table 1.

The pure breeding brown-seeded self-incompatible Canadian B. rapa variety ‘SPAN’ was crossed with the pure breeding yellow sarson self-compatible Bangladeshi B. rapa variety ‘BARI-6’ and the FI was backcrossed with ‘BAR1-6’. The F1, F2, F3 and BC1 were grown in a greenhouse at the University of Manitoba. A total of 224 F2 and 197 BC1 plants were used for seed coat color segregation analysis and molecular marker development for the seed coat color trait.

DNA was extracted using a modified CTAB method according to Li & Quiros (2001) from the flower buds of parental lines and their segregating populations. SRAP PCR amplification was the same as that of Li & Quiros (2001). Instead of autoradiography for signal detection, a five fluorescent dye set including, 6-FAM (blue), VIC (green), NET (yellow), PET (red), and LIZ (orange) supplied by Applied Biosystems (ABI), was used to separate SRAP PCR products with an ABI 3100 Genetic Analyzer (ABI, California).

The chromosome walking method is commonly used to determine genomic sequence flanking the know sequence of molecular markers. Siebert et al. (1995) have described a chromosome walking method on uncloned human genomic DNA, which was commercialized by Clontech Laboratories (Clontech, Mountain View, Calif.). The Genome Walker™ Universal Kit was used to obtain flanking chromosome sequence of the molecular marker linked to seed coat color. The procedure was performed according to the protocol provided in the Clontech kit. Genomic DNA of ‘SPAN’ (brown seeded parent) was digested with restriction enzymes Drat, EcoRV, Pvull and Stul. Sharp and strong bands were obtained after a second PCR amplification. These bands were excised from an agarose gel and DNA was extracted using a Qiagen Gel Extraction kit. All the DNA fragments were sequenced using a BigDyeerminator v1.1 Cycle Sequencing Kit.

SNP primer (GTGGTTGAGCGCTCAGTTGCA) (SEQ ID No. 15) and SCAR primers used in this study were designed using the Primer3 software. SNPs were detected with an ABI SNaPshot kit (ABI, Toronto). Genomic DNA was amplified first with specific primers targeting the corresponding SNP mutations. The PCR reaction was set up in 10 μl of reaction mix containing 60 ng of genomic DNA, 0.375 μM dNTP, 0.15 μM of each primer, 1×PCR buffer, 1.5 mM MgCl2 and 1 unit Taq polymerase. The PCR running program was 94° C. for 3 min, followed by 35 cycles of 94° C. for 1.0 min, 55.0 for 1.0 min, 72° C. for 1.0 min and final extension at 72° C. for 10 min. The amplified fragments were further analyzed with SNP detection primers and SNaPshot was performed according to the protocol in the ABI kit. The final products were separated with an ABI 3100 Genetic Analyzer. All four ddNTPs were fluorescently labeled with a different color dye i.e. the nucleotide ‘C’ was black, ‘T’ was red, ‘G’ was blue and ‘A’ was green. The alleles of a single marker were identified by different fluorescence color peaks after the data was analyzed with ABI GeneScan software.

The forward primer MR13 (TGCTCGTTCTTGACAACAC) (SEQ ID No. 16) and the reverse primer MR54 (GAGAATTGAGAGACAAAGC) (SEQ ID No. 17) were designed to target a deletion mutation that occurred in the black-seeded lines. To detect this deletion with the ABI 3100 Genetic Analyzer, an M13 primer sequence (CACGACGTTGTAAAACGAC) (SEQ ID No. 18) was added to the 5′ primer end of MR13 to create a primer MR1313 (CACGACGTTGTAAAACGACTGCTCGTTCTTGACAACAC) (SEQ ID No. 19). The M13 primer was labeled with four fluorescence dyes, 6-PAM, VIC, NED, and PET supplied by the ABI Company. In the PCR amplification, four different PCR reactions were set by four fluorescently labeled primers with separately unlabeled MR1313 and MR54 primers. The PCR reactions were mixed together in a 10 μl volume containing 60 ng of genomic DNA, 0.375 μM dNTP, 0.10 μM of M13 primer, 0.05 μM of MR1313 primer, 0.10 μM of MR54 primer, 1×PCR buffer, 1.5 mM MgCl2 and 1 Unit Taq polymerase. PCR was performed at 94° C. for 3 min, six cycles at 94° C. for 50 sec, 60° C. for 1.0 min 15 with a 0.7° C. decrease of annealing temperature at each cycle, 72° C. for 1.0 min, and then twenty cycles at 94° C. for 30 sec, 56° C. for 30 sec, 72° C. for 1.0 min for denaturing, annealing and extension, respectively, The PCR amplification products from different dye colors were pooled together so that each well contained four different fluorescently labeled DNA fragments which were detected in ABI 3100 Genetic Analyzer.

While the preferred embodiments of the invention have been described above, it will be recognized and understood that various modifications may be made therein, and the appended claims are intended to cover all such modifications which may fall within the spirit and scope of the invention.

### REFERENCES

Agren and Schemske, 1992, Artificial selection on trichome number in Brassica rapa. Theor Appl Genet 83:673-678
Ahmed S. U., Zuberi M. I. (1971) Inheritance of seed coat color in Brassica campestris L. variety Toria. Crop Sci 11:30
Baudry A, Caboche M, Lepiniec L. (2006) TT8 controls its own expression in a feedback regulation involving TTG1 and homologous MYB and bHLH factors, allowing a strong and cell-specific accumulation of flavonoids in Arabidopsis thaliana. Plant J. 46(5):768-79.
Baudry A, Heim M A, Dubreucq B, Caboche M, Weisshaar B, Lepiniec L. Broun, P., 2004, TT2, TT8, and TTG1 synergistically specify the expression of BANYULS and proanthocyanidin biosynthesis in Arabidopsis thaliana. Plant J. 39: 366-380
Broun, P., (2005) Transcriptional control of flavonoid biosynthesis: a complex network of conserved regulators involved in multiple aspects of differentiation in Arabidopsis. Curr. Opin. Plant Biol. 8, 272-279.
Chen B. Y., Jorgensen R. B., Cheng B. F., Heneen W. K. (1997) Identification and chromosomal assignment of RAPD marker linked with a gene for seed coat color in a Brassica campestris-alboglabra addition line. Hereditas 126:133-138.
Gruber M Y, Wang S, Ethier S, Holowachuk J, Bonham-Smith P C, Soroka J, Lloyd A. 2006 “HAIRY CANOLA”—Arabidopsis GL3 induces a dense covering of trichomes on Brassica napus seedlings. Plant Mol Biol. 60(5):679-98.
Johnson, C. S., Kolevski, B., and Smyth, D. R. (2002). TRANSPARENT TESTA GLABRA2, a trichome and seed coat development gene of Arabidopsis, encodes a WRKY transcription factor. Plant Cell 14, 1359-1375.
Lander E., Green P., Abrahamson J., Barlow A., Daley M., Lincoln S., Newburg L. (1987) MAPMAKER: an interactive computer package for constructing primary genetic linkage maps of experimental and natural populations. Genomics 1:174-181.
Li G, Quiros C F (2001) Sequence related amplified polymorphism (SRAP) a new marker system based on a simple PCR reaction: its application to mapping and gene tagging in Brassica. Theor Appl Genet 103:455-461
Liu H. L. (1992) Studies on inheritance of yellow-seeded Brassica napus L. Acta Agron Sin 18:241-249.
Liu Z. W., Fu T. D., Tu J. X., Chen B. Y. (2005) Inheritance of seed color and identification of RAPD and AFLP markers linked to the seed color gene in rapeseed (Brassica napus L.). Theor Appl Genet 110:303-310.
Michelmore R W, Paran I, Kesseli R V. 1991 Identification of markers linked to disease-resistance genes by bulked segregant analysis: a rapid method to detect markers in specific genomic regions by using segregating populations. Proc Natl Acad Sci USA. 1991 Nov. 1; 88(21):9828-9832
Negi M. S., Devic M., Delseny M., Lakshmikumaran M. (2000) Identification of AFLP fragments linked to seed coat color in Brassica juncea and conversion to SCAR marker for rapid selection. Theor Appl Genet 101:146-152.
Paterson A. H., Lan T. H., Amasino R., Osborn T. C., Quiros C. (2001) Brassica genomics: a complement to, and early beneficiary of, the Arabidopsis sequence. Genome Biol. 2: REVIEWS1011.1.
Rahman M. N. (2001) Production of yellow-seeded through interspecific crosses. Plant Breed 120:463-472
Joseph Sambrook and David W. Russell, 2001, Molecular cloning Cold Spring Harbor Laboratory Press, Cold Spring Harbor, N. Y., USA
Schiefelbein J., (2003) Cell-fate specification in the epidermis: a common patterning mechanism in the root and shoot. Curr Opin Plant Biol. 6:74-8
Somers D. J., Rakow G., Prabhu V. K., Friesen K. R. D. (2001) Identification of a major gene and RAPD markers for yellow seed coat color in B. napus. Genome 44:1077-1082.
Song, K. M., M. K. Slocum, and T. C. Osborn. 1995. Molecular marker analysis of genes controlling morphological variation in Brassica rapa. Theor. Appl. Genet. 90: 1-10.
Stringam G. R. (1980) Inheritance of seed color in turnip rape. Can J Plant Sci 60:331-335.
Stringam G. R., McGregor D. I., Pawlowski S. H. (1974) Chemical and morphological characteristics associated with seed coat color in rapeseed. In: Proc 4th Int Rapeseed Conf. Giessen, pp 99-108.
Tadanori Nozaki, Akira Kumazaki, Takato Koba, Keiko Ishikawa and Hiroshi Ikehashi, 1997, Linkage analysis among loci for RAPDs, isozymes and some agronomic traits in Brassica campestris L. Euphytica 95: 115-123
Tang Z. L., Li J. N., Zhang X. K., Chen L., Wang R. (1997) Genetic variation of yellow-seeded rapeseed lines (Brassica napus L.) from different genetic sources. Plant Breed 116:471-474.
Teutonico R. A., Osborn T. C. (1994) Mapping of RFLP and quantitative trait loci in Brassica rapa and comparison to the linkage maps of B. napus, B. oleracea and Arabidopsis thaliana. Theor Appl Genet 89:885-894.
Walker, A. R., Davison, P. A., Bolognesi-Winfield, A. C., James, C. M., Srinivasan, N., Blundell, T. L., Esch, J. J., Marks, M. D., and Gray, J. C. (1999). The TRANSPARENT TESTA GLABRA1 locus, which regulates trichome differentiation and anthocyanin biosynthesis in Arabidopsis, encodes a WD40 repeat protein. Plant Cell 11, 1337-1350.
Woo, S. S., J. Jiang, B. S. Gill, A. H. Paterson and R. A. Wing, 1994 Construction and characterization of a bacterial artificial chromosome library of Sorghum bicolor. Nucleic Acids Res. 22:4922-4931.
Adam-Blondon A F, Sevignac M, Bannerot H, Dron M (1998) SCAR, RAPD and RFLP markers linked to a dominant gene (Are) conferring resistance to a nthracnose in common bean. Theor Appl Genet 88:865-870
Barret P, Delourme R, Foisset N, Renard M (1998) Development of a SCAR (sequence characterized amplified region) marker for molecular tagging of the dwarf BREIZH (Bzh) gene in 1 Brassica napus L. Theor Appl Genet 97:828-833
Bradeen J M, Simon P W (1998) Conversion of an AFLP fragment linked to the carrot Y2 locus to a simple, co-dominant, PCR based marker form. Theor Appl Genet 97:960-967
Devic M, Albert S, Delseny M, Roscoe T J (1997) Efficient PCR walking on plant genomic DNA. Plant Physiol Biochem 35: 331-339
Heneen W K, Jorgensen R B (2001) Cytology, RAPD, and seed color of progeny plants from Brassica rapa-alboglabra aneuploids and development of monosomic addition lines. Genome 44:1007-1021
Jonsson R (1975) Yellow-seeded rape and turnip rape. II. Breeding for improved quality of oil and meal in yellow-seeded materials (in Swedish with English summary). Sveriges Utsadesforenings Tidsk rift 85:271-278
Karp A, Kresovich S, Bhat K V, Ayad W G, Hodgkin T (1997) Molecular tools in plant genetic resources conservation: a guide to the technologies; in IPGRI Technical Bulletin No. 2. International Plant Genetic Resources Institute, Rome, Italy
Lahogue F, This P, Bonquet A (1998) Identification of a co-dominant SCAR marker linked to the seedlessness character in grapevine. Theor Appl Genet 97: 950-959
Liu H L, Han J X, Hu X J (1991) Studies on the inheritance of seed coat colour and other related characteristics of yellow seeded Brassica napus. Proceedings of the 8th International Rapeseed Congress, Saskatoon, Canada, Vol. 5:1438-1444
Mahmood T, Rahman M H, Stringam G R, Raney P J, Good A G (2005) Molecular markers for seed color in Brassica juncea. Genome 48:755-760
Mohammad A, Sikka S M, Aziz M A (1942) Inheritance of seed colour in some oleiferous Brassiceae. Indian J Genet 2:112-127
Naqvi N I, Chattoo B B (1996) Development of a sequence characterized amplified region (SCAR) based indirect selection method for a dominant blast resistance gene in rice. Genome 39:26-30
Nirupma Pati, Valerie Schowinsky, Obrad Kokanovic, Victoria Magnuson, Soumitra Ghosh (2004) A comparison between 1 SNaPshot, pyrosequencing, and biplex invader
SNP genotyping methods: accuracy, cost, and throughput. J Biochem Biophys Methods 60:1-12
Padmaja K L, Arumugam N, Gupta V, Mukhopadhyay A, Sodhi Y S, Pental D, Pradhan A K (2005) Mapping and tagging of seed coat colour and the identification of markers for marker-assisted manipulation of the trait in Brassica juncea microsatellite. Theor Appl Genet 111(1):8-14
Rahman M H, Joersbo M, Poulsen M H (2001) Production of yellow-seeded Brassica napus of double low quality. Plant Breeding 120:473-478
Schwetka A (1982) Inheritance of seed color in turnip rape (Brassica campestris L.). Theor Appl Genet 62:161-169
Shirzadegan M, Robbelen G (1985) Influence of seed colour and hull proportions on quality properties of seeds in Brassica napus L. Fette Seifen Anstrichm 87:235-237
Shirzadegan M (1986) Inheritance of seed coat color in Brassica napus L. Z Pflanzenzuecht 96:140-146
Siebert P D, Chenchik A, Kellogg D E, Lukyanov K A, Lukyanov S A (1995) An improved method for walking in uncloned genomic DNA. Nucleic Acids Res 23:1087-1088
Van Deynze A E, Landry B S, Pauls K P (1995) The identification of restriction fragments length polymorphisms linked to seed color genes in Brassica napus. Genome 38:534-542
Vos P, Hogers R, Bleeker M, Reijans M, van de Lee T, Homes M, Freijters A, Pot J, Peleman J, Kuiper M, Zabeau M (1995) AFLP a new technique for DNA fingerprinting. Nucleic Acids Res 23:4407-4414
Williams G K, Kubelik A R, Livak K J, Rafalski J A, Tingey S V (1990) DNA polymorphisms amplified by arbitrary primers are useful as genetic markers. Nucleic Acids Res 18:6531-6535
Zaman M W (1989) Inheritance of seed colour in Brassica campestris. Sveriges Utsadesfdrenings Tidskrift 99:205-207
Zhi-wen Liu, Ting-dung Fu, Jin-xing Tu, Bao-yuan Chen (2005) Inheritance of seed color and identification of RAPD and AFLP markers linked to the seed color gene in rapeseed (Brassica 1 napus L.). Theor Appl Genet 110(2):303-310
Zhi-wen Liu, Tingdong Fu, Ying Wang, Jinxing Tu, Baoyuan Chen, Yongming Zhou, Chaozhi Ma, Lianmin Shan (2006) Development of SCAR and CAPS markers for a partially dominant yellow seed coat gene in Brassica napus L. Euphytica 149:381-385

TABLE 1
Primers for SCAR markers
and screening B. rapa BAC library

| | | |
|---|---|---|
| JF39 | CCGCATGTTTCACCAACC | (SEQ ID No. 7) |
| JF40 | TGGCCTTACATAGTGGAAG | (SEQ ID No. 8) |
| JF5G3 | ATAGAAAGTAAAGGTACTCTCTT | (SEQ ID No. 9) |
| JF5G4 | GGTACTCTCTTTTTAGTGCGA | (SEQ ID No. 10) |
| JF5G5 | ACCAGTTCCTTGTTCGTTC | (SEQ ID No. 11) |
| JF87a | GTCCCAACCTGCGTTCTA | (SEQ ID No. 12) |
| JF106 | CAGAGCATAAATCTCCTGC | (SEQ ID No. 13) |
| JF106b | CCAGAGCATAAATCTCTTATG | (SEQ ID No. 14) |

1. A gene-silencing construct comprising at least 20 consecutive nucleotides of the Brassica TTG1 (SEQ ID Nos: 1-3) sequence or TT1 sequence (SEQ ID Nos. 4-5).

2. A method of controlling seed color comprising:
transforming a plant with a gene-silencing construct comprising at least 20 consecutive nucleotides of the Brassica TTG1 (SEQ ID Nos: 1-3) sequence or TT1 sequence (SEQ ID Nos. 4-5);
growing the plant under conditions whereby the gene silencing construct is expressed, thereby interfering with native TTG1 or TT1 expression such that said plant produces yellow seeds.

3. The method according to claim 2 wherein the plant is a Brassica species.

4. A construct for producing yellow seeds in a Brassica plant comprising the seed-specific tt8 promoter (SEQ ID No. 6) operably linked to Brassica TTG1 (SEQ ID No. 2) or TT1 (SEQ ID No. 4).
